(19) United States
(12) Patent Application Publication
(10) Pub. No.: US 2005/0125058 A1
(43) Pub. Date: Jun. 9, 2005

(54) Accommodating hybrid intraocular lens

(75) Inventors: J. Stuart Cumming, Laguna Beach, CA (US); Gerardo Lugo, Alta Loma, CA (US); Jonathan R. Soiseth, Pomona, CA (US)

Correspondence Address: ORRICK, HERRINGTON & SUTCLIFFE, LLP, 4 PARK PLAZA, SUITE 1600, IRVINE, CA, 92614-2558, US

(73) Assignee: eyeonics, inc.

(21) Appl. No.: 10/888,536

(22) Filed: Jul. 8, 2004

Related U.S. Application Data
(60) Provisional application No. 60/527,337, filed on Dec. 3, 2003.

(51) Int. Cl. A61F 2/16

(52) U.S. Cl. 623/6.37; 623/6.44; 623/6.46; 623/6.51

(57) ABSTRACT

The hybrid accommodating intraocular lens comprises an optic made from a flexible material combined with extended portions made of a second flexible material that is capable of multiple flexions without breaking.

This application claims priority to U.S. Provisional Application Ser. No. 60/527,337, filed on Dec. 3, 2003. Priority to the prior application is expressly claimed, and the disclosure of the application is hereby incorporated by reference in its entirety.

### BACKGROUND

Intraocular lenses have for many years had a design of a single optic with loops attached to the optic to center the lens and fixate it in the empty capsular bag of the human lens. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone.

In the mid 1990s an acrylic material was introduced as the optic of lenses. The acrylic lens comprised a biconvex optic with a straight edge into which were inserted loops to center the lens in the eye and fixate it within the capsular bag. Claims were made that the material of this lens significantly reduced posterior capsular opacification. It later became apparent that the property of the lens that reduced posterior capsular opacification was not necessarily related to the material, but the relatively sharp or “square edge” on the posterior surface of the optic. The optic, when it is sealed within the capsular bag, presented a square sharp edge to the posterior capsule, which is tightly pulled against it during the period of fibrosis, preventing the ectodermal cells from growing across the posterior capsule behind the optic. A barrier was formed which successfully reduced the posterior capsular opacification rate. Silicone optic lenses were manufactured with a similar so-called straight edge on the posterior surface of the optic. Studies were done and the instance of posterior capsular opacification was found to be the same in the silicone lenses as in the acrylic lenses; therefore the material was not the cause of the reduction in posterior capsular opacification. The sharp edge of the optic where the posterior optic surface joined the edge of the lens was the prime reason for the reduced incidence of posterior capsule opacification.

Recently accommodating intraocular lenses have been introduced to the market, which generally are modified plate haptic lenses and, like the silicone plate haptic lenses, have no clear demarcation between the junction of the plate with the optic's posterior surface. A plate haptic lens may be defined as an intraocular lens having two or more plate haptics where combined junctions with the optic represent one quarter or more of the circumference of the optic.

### SUMMARY OF THE INVENTION

According to a preferred embodiment of this invention, an accommodating lens comprises a hybrid lens with a flexible acrylic optic attached to which are two or more extended portions which may be plate haptics capable of multiple flexions without breaking, along with fixation and centration features at their distal ends. There may be a hinge across the extended portions adjacent to the optic to facilitate the anterior and posterior movement of the optic relative to the outer ends of the extended portions.

Flexible acrylic material has gained significant popularity among ophthalmic surgeons. In 2003 more than 50% of the intraocular lenses implanted had acrylic optics. Hydrogel lenses have also been introduced. Both the acrylic and hydrogel materials are incapable of multiple flexions without fracturing.

The advent of an accommodating lens which functions by moving along the axis of the eye by repeated flexions somewhat limited the materials from which the lens could be made. Silicone is the ideal material, since it is flexible and can be bent probably several million times without showing any damage. Additionally a groove or hinge can be placed across the plate adjacent to the optic as part of the lens design to facilitate movement of the optic relative to the outer ends of the haptics. On the other hand, acrylic material fractures if it is repeatedly flexed.

The purpose of this invention is to provide a hybrid lens that has a flexible and foldable acrylic or hydrogel optic preferably with a 360 degree sharp posterior edge, and plate haptics that are capable of multiple flexions without breaking and which are attached to the acrylic or hydrogel optic. Fixation features attached to the distal ends of the plates help center the lens and fixate the lens in the capsular bag.

### BRIEF DESCRIPTION OF THE DRAWINGS

FIG. 1 illustrates a preferred embodiment of the present invention comprising a perspective view thereof in FIG. 1a, front elevational view in FIG. 1b, side elevational view in FIG. 1c, cross-sectional view in FIG. 1d along the line of d-d of FIG. 1b, a detailed view in FIG. 1f of area b in FIG. 1c, and a perspective view in FIG. 1e of a Kapton ring of this embodiment. FIG. 1g is a further view of an embodiment using a ring surrounding a groove in the optic to hold the optic securely in place using different materials and particular design from that shown in FIGS. 1a-1f.

FIG. 2 shows two different ways of fixating the flexible plates to the optic whereby a projection can extend from the optic material into the plate material and acts as an anchor as seen to the left. The right-hand side of FIG. 2 shows an alternative of a projection from the flexible plate into the optic to fixate the plate to the optic.

FIGS. 3a-3e illustrate the manner in which fixation devices can be attached to a loop. FIG. 3a shows an inlay, which may be of polyimide, prolene, or PMMA inserted into the flexible loop, the loop being of the same material as the plate; FIGS. 3b-3d show another variation, whereby the fixation device is slideable along the length of the flexible loop and kept from disengaging by means of a small peg in a slot within the flexible loop (3b); FIG. 3c shows an alternative fixation device; and FIG. 3e is a drawing of the fixation device before insertion in the loop.

FIGS. 4a-4b show other embodiments and preferred attachments.

FIGS. 5-17 show further variations, and FIG. 5 is an elevational view of a lens having plate haptics extending oppositely from the optic and having a flexible optic of one material with flexible plate haptics of another material, the plate haptics having hinges or grooves across their plates adjacent to the optic.

FIG. 6 is a cross section of FIG. 5 showing the hinges or grooves across the haptics.

FIG. 7 is another version of the lens illustrating an optic of one material and the plate haptics of another with fixation devices on the end of the plate haptics comprising projections on the anterior, posterior, or both surfaces at the distal ends of the plates.

FIG. 8 is a cross section of FIG. 7.

FIG. 9 is another version with different fixation devices extending from the corners of each of the plates.

FIG. 10 is an elevational view of yet another version of the accommodating hybrid lens with an acrylic or hydrogel optic and silicone plate haptics fixated to the optic and flexible loops of a different material extending from the ends of the plates.

FIG. 11 is similar to FIG. 10 except that there are no hinges across the plates as shown in FIG. 10, the haptics being sufficiently resilient such that they do not require hinges.

FIG. 12 shows an optic of flexible material to which is attached four radially extending plate haptics engaging a rim in the optic.

FIG. 13 is another variation of a hybrid lens with two half-discs and fixation devices at their periphery.

FIG. 14 is a complete disc surrounding a hybrid optic with fixation and centration devices attached to the rim of the disc.

FIG. 15 shows yet another embodiment of a lens, showing T-shaped haptics engaged in the capsular bag having been depressed by the bag wall towards the optic. Hinges are across the plate haptic adjacent to the optic.

FIG. 16 illustrates the movement of the T-shaped haptics of FIG. 15 to match the inside diameter of the human capsular bag. The T-shaped haptics may be integral to the plate or may be of a separate material from the plate.

FIG. 17 shows T-shaped haptics where the arm of the haptic is composed of the same material as the plate and on the end of the haptic there is an inlay of a different material which is designed to anchor the T-shape within the capsular bag.

FIG. 18 shows an alternative embodiment.

According to the present invention the optic is of a foldable, flexible acrylic or hydrogel material and the haptic plates are of a foldable material that will withstand multiple foldings without damage, e.g., silicone. Preferably, the end of the plate haptics have T-shaped fixation devices and are hinged to the optic.

### DESCRIPTION OF THE PREFERRED EMBODIMENTS

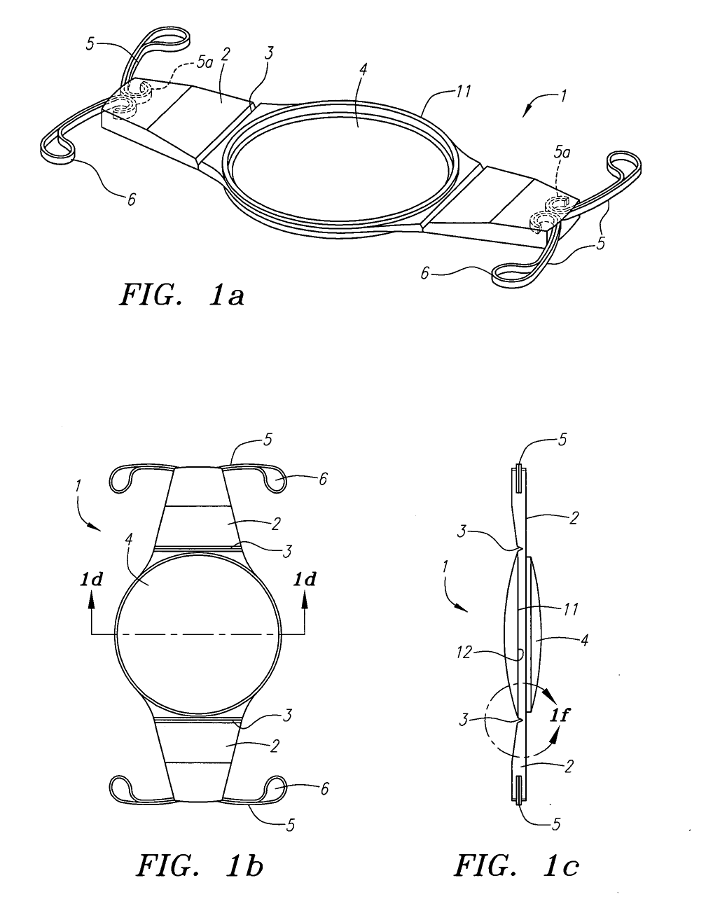

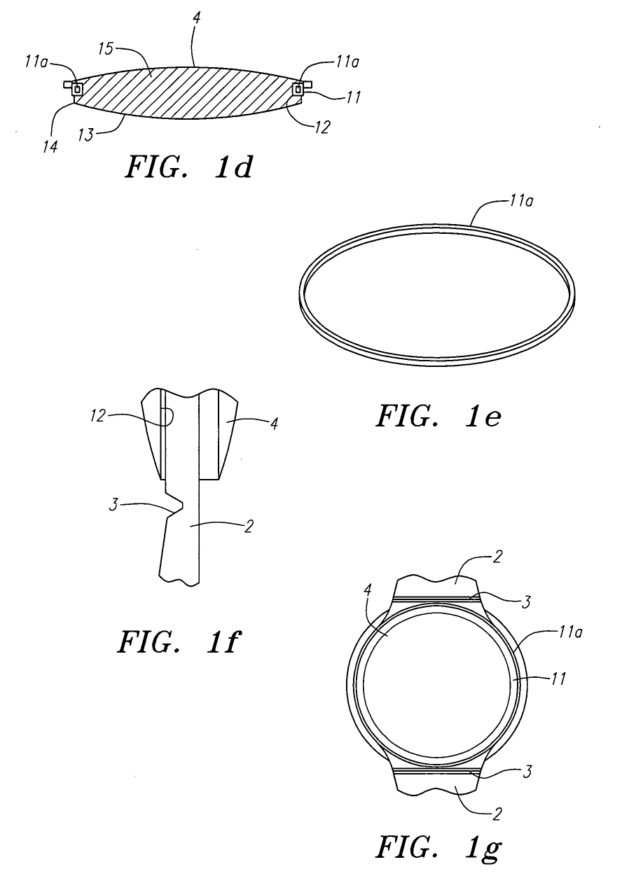

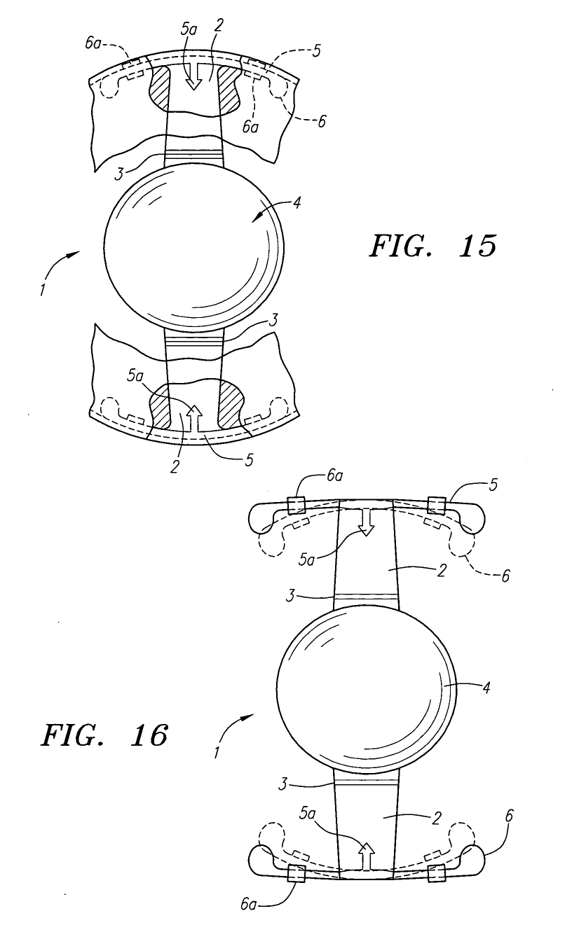

Turning now to FIG. 1, a preferred embodiment is illustrated in greater detail comprising an intraocular lens 1 with which the hybrid nature of the lens 1 is formed of two components, namely, a flexible optic 4 preferably made of acrylic or hydrogel which is incapable of multiple flexations, and flexible extending portions which may be plate haptics 2 which are capable of multiple flexations without damage, formed, for example, of silicone. The optic 4 and plate haptics 2 preferably are uniplanar. One or more plate haptics 2 extend distally from opposite sides of the optic 4. In this embodiment, the haptics are plate haptics having arcuate outer edges including loops 5. The loops 5 when unrestrained are somewhat less curved in configuration as shown in FIGS. 1a-1b (but compare an example of an inserted lens 1 as seen in FIG. 15). The lens 1, including the optic 4, haptics 2, and loops 5 is preferably formed of a semi-rigid material such as silicone, acrylic, or hydrogel, and particularly a material that does not fracture with time. The loops 5 can be of a material different from the haptics 2 and retained in the haptics by loops 5a molded into the ends of the haptics.

In the embodiment shown in FIG. 1, the flexible haptics 2 and loops 5 preferably are connected to the optic 4 by means of an encircling elastic band 11 which fits into a groove 12 in the optic 4 as seen in FIGS. 1c-1d & if, the two components being manufactured separately and assembled after manufacture by slipping the flexible haptic band 11 into the groove 12 in flexible optic 4. Alternatively and preferably, the band 11 which may be of silicone includes an internal ring 11a formed of polyimide, nylon, or PMMA, and the haptics 2 are formed of silicone.

The groove 12 is located in the edge of the optic 4 as seen in FIG. 1d such that there is a sharp edge 14 on the posterior surface 13 of the optic 4. The junction of the posterior surface 13 of the optic 4 to the edge of the lens 1 is a sharp edge or junction 14 designed to reduce the migration of cells across the posterior capsule of the lens post-operatively and thereby reduce the incidence of posterior capsular opacification and the necessity of YAG posterior capsulotomy. The anterior surface 15 of the optic 4 is closer to the groove 12 than is the posterior surface 13. The circular band 11 (see FIGS. 1a-1c) is stretchable and is manufactured to be smaller than the diameter of the optic 4 such that it can be slipped over the optic to tightly engage in the rim or groove 12 around the optic 4.

FIG. 1 diagrammatically illustrates the elastic haptics 2, loops 5, hinge 3 across the haptics adjacent to the optic, and shows the circular elastic molding that engages the groove around the periphery of the optic 4. A hard knob 6 can be provided on the ends of the loops 5 and designed to fixate the loops in the capsular bag and at the same time allow the elastic loop 5 to stretch along its length as the optic of the lens moves backwards and forwards and the plate haptics 2 move or slide within pockets formed between the fusion of the anterior and posterior capsules of the capsular bag.

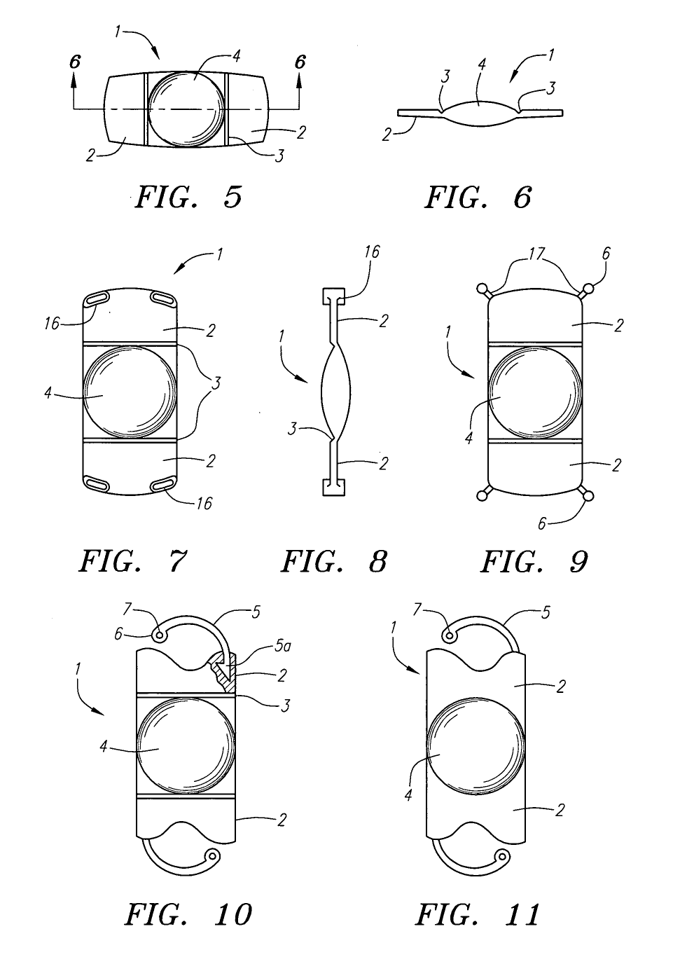

The various Figures disclose several forms of lenses to which the present concepts are applicable. The general configurations as shown in FIGS. 5 through 16 are to some extent similar to lenses shown in Cumming U.S. Pat. Nos. 5,476,514, 6,051,024, 6,193,750, and 6,387,126, except for the incorporation into lenses of those general configurations the concepts of the present invention particularly of providing a hybrid lens with a flexible acrylic optic attached to which are two or more haptics capable of multiple flexations without breaking, along with suitable fixation and/or centration features at the distal ends of the haptics. Forms of lenses 1 are shown with haptics 2, an optic 4, in some cases hinges 3 or grooves across the haptics 2 adjacent to the optic 4 and, further, in some cases loops 5, knobs 6, and notches 7.

As is well known in the art, intraocular lens 1 such as that in the drawings is implanted in the capsular bag of the eye after removal of the natural lens. The lens is inserted into the capsular bag by a generally circular opening cut in the anterior capsular bag of the human lens and through a small opening in the cornea or sclera. The outer ends of the haptics 2, or loops 5, are positioned in the cul-de-sac of the capsular bag. The outer ends of the haptics, or the loops, are in close proximity with the bag cul-de-sac, and in the case of any form of loops, the loops are deflected from the configuration as shown for example in FIG. 16 to the position shown in FIG. 15. Knobs 6 can be provided on the outer end portions of the loops for improved securement in the capsular bag or cul-de-sac by engagement with fibrosis, which develops in the capsular bag following the surgical removal of the central portion of the anterior capsular bag. The end of the loops containing the knobs may be either integrally formed from the same material as the haptic plates or loops or may be of a separate material such as polyimide, prolene, or PMMA as discussed below. The loop 5 can be integrally formed with the plate or the loop may be formed of a separate material. As will be discussed in further detail subsequently, the loops formed of a separate material are molded into the terminal portions of the haptics such that the flexible material of the loop can extend by elasticity along the internal fixation member of the loop. As noted above, the haptics plates 2 may have a groove or a hinge 3 across their surface adjacent to the optic. This facilitates movement of the optic anteriorly and posteriorly relative to the outer ends of the haptics.

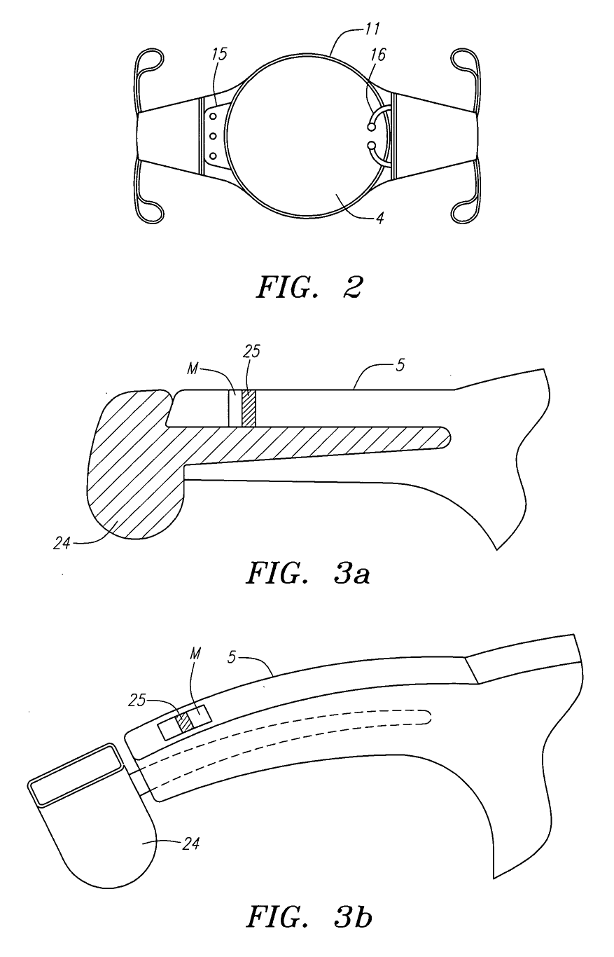

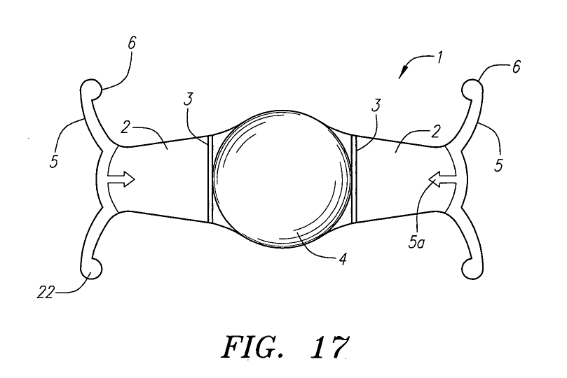

Returning to FIGS. 5 through 17, FIG. 5 shows a simple plate lens design with hinges 3 across the haptic plates 2 and the optic 4 formed of a separate material. The plates 2 can engage the optic 4 in the manner illustrated in FIG. 2 by means of projections 15 from the optic 4 into the plate 2 or alternatively from projections 16 from the plate 2 into the optic 4, or preferably by the circumferential groove 12 in the edge of the optic 4 (FIG. 1d) into which fits the circular elastic ring molded as part of the two plates.

FIG. 7 shows the hinge 3 across the plate 2, and illustrates a different hybrid lens where the fixation devices compromise protrusions 16 which may protrude anteriorly or posteriorly, or both anteriorly and posteriorly, from the distal ends of the haptics 2. There may be several of these protrusions 16 on each haptic. FIG. 8 shows a cross section of FIG. 7.

FIG. 9 shows an additional way of producing a hybrid lens where the fixation devices are knobs 6 on the ends of small arms 17 at the corners of each of the plate haptics 2.

FIG. 10 shows another accommodating lens whereby the fixation and centration devices are formed by arcuate loops 5 that are molded into the plate haptics 2. The plate haptics in FIGS. 5 through 11 are designed to attach to the optic 4, which is of a different material than the plates. In FIG. 11 there is no groove across the plates 2, the plates being sufficiently flexible in this embodiment that a hinge mechanism is not required. Holes 7 also can be provided.

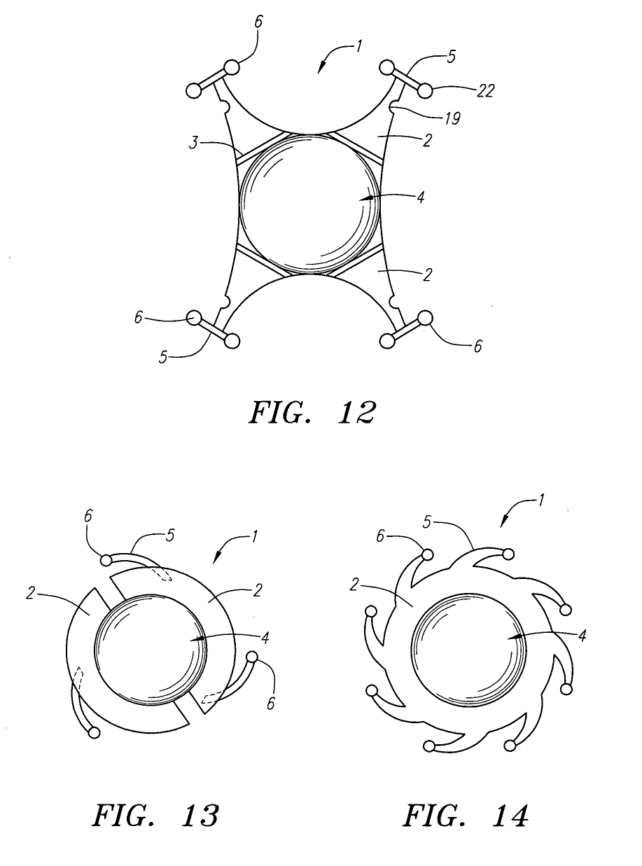

FIG. 12 shows another variation of the lens 1 with four haptic plates 2 and hinges 3, along with a T-section at the end of each plate 2 and with knobs 6 on the ends for fixation in the eye. There also can be notches 19 in the plates 2 to further enhance the fixation of the distal part of the plates into the eye.

FIG. 13 shows a disc variation of a hybrid lens with two half plates 2, with loops 5, and a knob 6 at the end of each loop for fixation and centration of the lens within the eye. The lens plates and optics are of different materials as previously discussed, preferably the plates being silicone and the optic being an acrylic material.

FIG. 14 shows another variation of the circular plate design with one or more centration and fixation devices about the periphery of the flexible plate, with a central optic 4 preferably made of acrylic.

FIGS. 15 and 16 illustrate a plate haptic 2 design with T-shaped flexible loops 5 extending from the corners of each of the plates 2. Hinges 3 facilitate the anterior and posterior movement of the optic relative to the outer ends of the plates. Collars 6a may be provided.

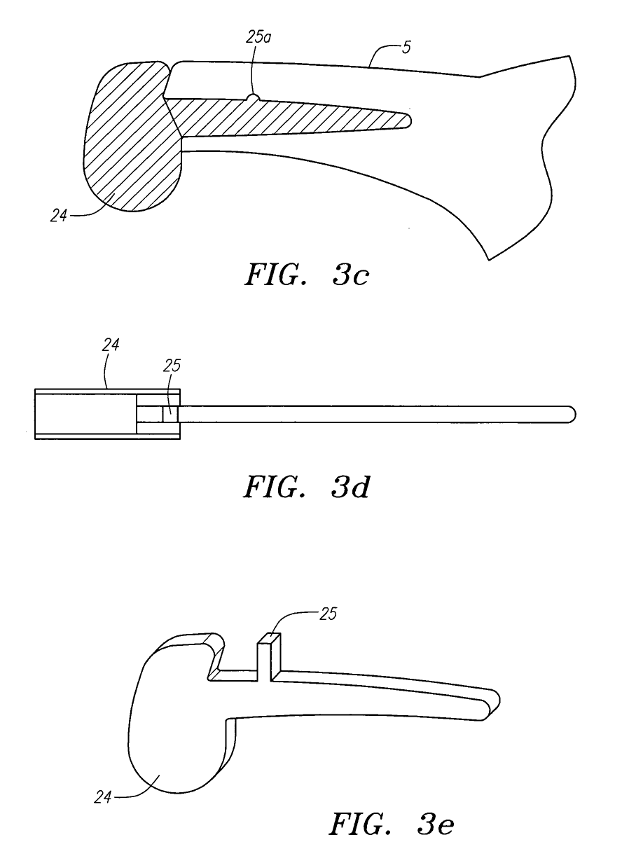

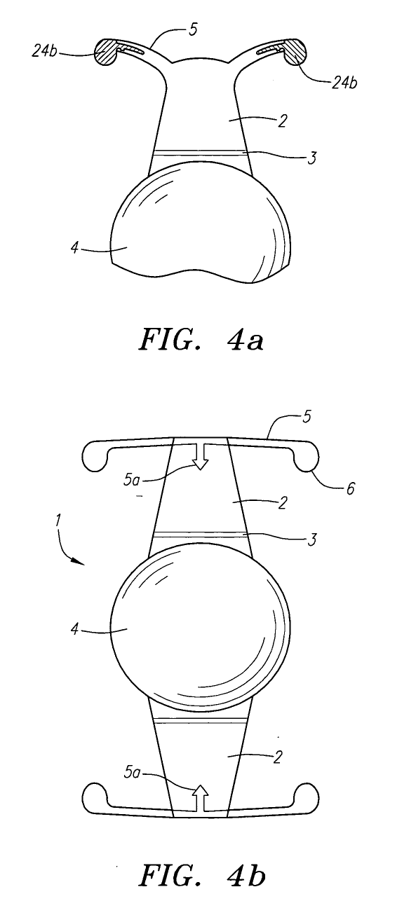

FIGS. 3a through 3e illustrate variations whereby the fixation device 24 is slideable partially along the length of the flexible loop 5 and maintained from disengaging by means of a small peg or tooth 25 in a slot M within the flexible loop or similar arrangement. An integral peg 25 of FIGS. 3b and 3d maintains the device 24 within the loop 5. Preferably, the fixation device 24 is of polyimide. FIG. 3b is a perspective view. FIG. 3c is a cross-sectional view of a variation with a tooth or bump 25a. FIG. 3e is a perspective view of a fixation device 24.

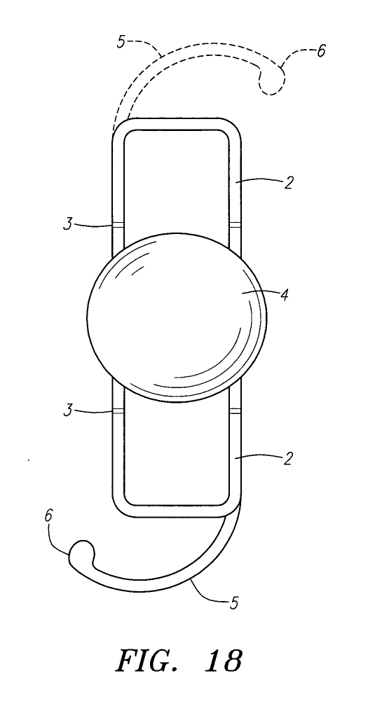

FIG. 18 illustrates an alternative embodiment wherein the extending portions or haptics are in the form of thin members 2, extending from the optic 4. Centration/fixation loops 5 can be added to both outer ends or not added as desired, and likewise hinges 3 as shown can be provided on both sets of haptics or omitted from both as desired. Furthermore, knobs 22 can be provided at the ends of loops 5, or omitted.

Thus there has been shown and described a hybrid lens that ideally comprises an acrylic optic and silicone plates with a fixation device at the end of the loops of a different material than the plate, allowing for movement of the loops along the tunnel formed in the fusion of the anterior and posterior capsules of the human capsular bag.

Various changes, modifications, variations, and other uses and applications of the subject invention will become apparent to those skilled in the art after considering this specification together with the accompanying drawings and claims. All such changes, modifications, variations, and other uses of the applications which do not depart from the spirit and scope of the invention are intended to be covered by the claims which follow.

1. An accommodating intraocular lens comprising a flexible optic which is of a different material than attached flexible extended portions, designed such that the optic can move backwards and forwards relative to the outer ends of the extended portions and may assume a position such that the optic can be in front of, in the same plane or behind the outer ends of the haptics and can achieve accommodation by one of (a) without the optic starting in a posterior position ever moving anterior to the outer ends of the extending portions, (b) without the optic starting in an anterior position ever moving posterior to the outer ends of the extending portions, and (c) by the optic moving from a posterior to an anterior or uniplanar position relative to the outer ends of the extending portions.

2. A lens according to claim 1 wherein said haptics comprise one or more plate haptics.

3. A lens according to claim 1 wherein one or more fixation devices are on one or more ends of the extended portions.

4. A lens according to claim 2 wherein the extended portions are plate haptics and there is a groove or hinge across one or more of the plate haptics adjacent to the optic.

5. A lens according to claim 1 where the optic is acrylic.

6. A lens according to claim 1 where the optic is a hydrogel.

7. A lens according to claim 1 where the extended portions are silicone.

8. A lens according to claim 1 where the extended portions may further include loops and fixation devices and may be a combination of silicone and another inert material, including polyimide, prolene, or PMMA.

9. A lens according to claim 1 including fixation devices comprising loops made from polyimide, PMMA, or prolene.

10. A lens according to claim 8 where the loops are of the same material as the plates.

11. A lens according to claim 10 where the loops have a fixation element of a different material on their proximal ends to enhance centration and fixation of the lens within the capsular bag.

12. A lens according to claim 1 where the optic size is from 3.5 to 8 mm.

13. A lens according to claim 1 where the attachment of the extended portions to the optic is by means of projections of the optic material into the flexible haptic.

14. A lens according to claim 1 where the attachment of the optic to the extended portions is by means of projections of the haptic material into the optic.

15. A lens according to claim 1 where the attachment of the extended portions to the optic is by means of an encircling band extending from the flexible haptics to encircle a groove 360 degrees around the periphery of the optic.

16. A lens according to claim 15 where the flexible haptics are flexible plates.

17. A lens according to claim 16 where the flexible plates have a groove across one or more of the plates adjacent to the optic.

18. A lens according to claim 16 wherein the extended portions have flexible fixation devices at their outer ends made from a different material.

19. A lens according to claim 16 wherein the extended portions comprise plates and loops and are made from the same material and further include fixation devices of a different material, including polyimide, prolene, or PMMA.

20. A lens according to claim 16 wherein the haptics comprise plates, loops, and fixation devices all from the same material.

21. A lens according to claim 15 wherein the encircling band is formed of silicone and includes an internal ring comprising one of polyimide, nylon or PMMA to prevent the band from dislocating from the optic.
